(19) United States
(12) Patent Application Publication
(10) Pub. No.: US 2023/0115584 A1
(43) Pub. Date: Apr. 13, 2023

(54) APPLICATION OF FULLERENE AND DERIVATIVES THEREOF IN REGULATING INTESTINAL FLORA

(71) Applicant: Xiamen Funano New Material Technology Co., Ltd., Xiamen, Fujian (CN)

(72) Inventors: Xiaoqing LI, Xiamen, Fujian (CN); Jiamei HUANG, Xiamen, Fujian (CN); Changfeng ZHU, Xiamen, Fujian (CN); Yanyu WU, Xiamen, Fujian (CN); Yaling LIU, Xiamen, Fujian (CN)

(21) Appl. No.: 17/909,638

(22) PCT Filed: Mar. 3, 2021
(86) PCT No.: PCT/CN2021/078828

§ 371 Date: Sep. 6, 2022

(30) Foreign Application Priority Data
Mar. 6, 2020 (CN) 202010149176.9

(51) Int. Cl. A61K 33/44; A61K 9/107; A61K 47/44; A61P 1/00; A61P 31/04

(52) U.S. Cl. CPC A61K 33/44; A61K 9/1075; A61K 47/44; A61P 1/00; A61P 31/04

(57) ABSTRACT

The present invention relates to the field of fullerene application, and in particular to an application of fullerene and a derivative thereof in regulating intestinal flora. The fullerene and the derivative thereof may increase beneficial bacteria, decrease harmful bacteria and increase the abundance of intestinal flora when used for regulating intestinal flora, which is thus capable of relieving or treating human diseases (including gastrointestinal diseases, neurodegenerative diseases, metabolic diseases, sleep disorders, inflammation, tumors) and enhancing human immunity, improving animal physiques, regulating animal intestinal tracts, preventing or treating animal diseases, or promoting animal growth with great application prospect.

### TECHNICAL FIELD

The present invention relates to the field of fullerene application, and in particular to an application of fullerene and a derivative thereof in regulating intestinal flora.

### BACKGROUND

A large number of symbiotic microorganisms are parasitic in animal intestinal tracts. These symbiotic microbial florae form the “second largest organ” of a body, and affect the health status of the host by participating in body's digestion and absorption, material metabolism, lipid reserve, inflammatory response and other process. Intestinal flora consists of bacteria, archaebacteria, fungi, virus and the like; the total number of flora can be up to 100 trillion and is 10 times the total number of body cells. Classified by Phylum, intestinal flora mainly includes Firmicutes, Proteobacteria, Actinobacteria, Bacteroidetes, Fusobacteria, Verrucomicrobia, Cyanophyta, Spirochaetes, and the like. The influences of intestinal flora on a host are not only limited to the intestinal system. The current studies have indicated that the imbalance of intestinal flora affects the occurrence and prognosis of lots of diseases. In animal colonitis, obesity and type-I diabetes models, the absence of host genes can lead to the change of intestinal microecology; a changed intestinal microecology is transferred or transmitted to another host, which is capable of changing the prognosis of diseases. The quantity and composition of flora in intestinal microecology can cause direct or indirect effects on the physiological functions of liver, and is likely to affect the occurrence and progression of a hepatic disease. The intestinal microecology is closely related to liver, and is likely to be bidirectional to host organs by bile, hormones, inflammatory mediators, digested and absorbed metabolites. The current studies have shown that bacteria in the microbial ecosystem is unfathomably associated with the health, beautifying, and even characters of a human body. In medical aspect, cancer, diabetes, neurodegenerative diseases, depressive disorders, infantile autism and the like are related to intestinal microecology.

Fullerene is a kind of C nanoatom cluster composed of 60-120 C atoms, and it is an allotrope of graphite and diamond, capable of affiliating free radicals and thus, has strong oxidation resistance. Due to biological activities, such as antiviral action, oxidation resistance and antibacterial action, fullerene has been widely applied in our daily life and social production, such as cosmetics, drug carriers, medical diagnosis and other numerous fields.

### SUMMARY

The present invention is aimed at providing an application of fullerene and a derivative thereof in regulating intestinal flora.

Through intensive study, the inventor of the present invention finds that fullerene and derivatives thereof can regulate the abundance of intestinal flora, increase the diversity of flora and balance the microecology of flora, thus ensuring the microecological balance of intestinal flora in the aspects of increasing beneficial bacteria and controlling harmful bacteria. Regulation on the abundance of intestinal flora is different from the effect on a single bacterial genus, and also is different from the regulation on the growth and metabolism of a category of or a microorganism. Intestinal flora is a complex system; and regulation on a single bacterial genus may not exactly explain the effects of fullerene and derivatives thereof on human health. 16S rRNA and metagenome are utilized for sequencing analysis to know that fullerene and derivatives thereof balance beneficial and harmful bacteria by regulating the abundance of intestinal flora, thus regulating the overall status of intestinal flora. Fullerene and derivatives thereof regulate and control intestinal flora in the levels of abundance and diversity such that beneficial bacteria, neutral bacteria and harmful bacteria in the intestinal tract are in a balanced state, which is different from probiotics or bactericides, namely, acting on intestinal flora only in the way of increasing beneficial bacteria or killing harmful bacteria. Intestinal flora environment varies from each individual, and a single beneficial bacterium also varies from each individual. The correlation of intestinal flora to diseases is not that a kind of flora affects a disease, but a disease is caused when the whole flora environment changes. Fullerene and derivatives thereof may affect flora microecology to make the intestinal tract healthier, thus achieving the effects of improving immunity and treating related diseases. The present invention is completed based on this.

Specifically, the present invention provides an application of fullerene and a derivative thereof in regulating intestinal flora.

Further, the fullerene and the derivative thereof are selected from at least one of C₂ₘ, a fullerene metal derivative, a fullerene oxygen-bearing derivative, and organic compound-modified or enveloped fullerene; oxygen atoms in the fullerene oxygen-bearing derivative are linked to carbon atoms or bonded to an alkylene chain on a fullerene framework; an organic compound in the organic compound-modified or enveloped fullerene is cyclodextrin and/or a crown ether, and 30≤m≤60.

Further, the fullerene and the derivative thereof represent as any one of C₂ₘ, M@C₂ₘ, M₂@C₂ₘ, MA@C₂ₘ, M₃N@C₂ₘ, M₂C₂@C₂ₘ, M₂S@C₂ₘ, M₂O@C₂ₘ and M_{X}A_{3-X}N@C₂ₘ, and 30≤m≤60; M and A are each independently selected from any one metallic element of Sc, Y and lanthanide elements, and @ is a linking group or a representing method of a metal.

Further, in use procedure, the fullerene and the derivative thereof are used as food, medicaments or health care products in a form of tablet, hard capsule, soft capsule, enteric capsule, micro-capsule powder, granule, syrup, injection, emulsion, suspending agent, aerosol, solution, oral or non-oral sustained-release agent, with or without other ingredients added.

According to one preferred embodiment of the present invention, the fullerene and the derivative thereof are used in the form of micro-capsule powder; at this time, the fullerene and the derivative thereof have better stability and may effectively overcome the shortcomings of fullerene compounds such as difficult preservation and easy oxidation, thus giving play to the efficacy of the fullerene compounds in regulating intestinal flora to the utmost extent. Components of the micro-capsule powder are fullerene and/or a fullerene derivative, a nutritious compound oil, an emulsifying agent, a wall material and an optional stabilizer. The nutritious compound oil is a complex of olive oil and flaxseed oil; the emulsifying agent is a complex of sodium caseinate and mono/di-glycerin fatty acid ester; the wall material is maltodextrin, and the stabilizer is dipotassium phosphate. Each component in the fullerene micro-capsule powder has the following amount: 1 g of fullerene and/or fullerene derivative, 0.5-1.5 L of nutritious compound oil, 5-15 g of sodium caseinate, 10-20 g of mono/di-glycerin fatty acid ester, 400-600 g of maltodextrin and 4-6 g of dipotassium phosphate; a weight ratio of the olive oil to the flaxseed oil in the nutritious compound oil is 5:5-7:3.

According to one preferred embodiment of the present invention, the micro-capsule powder is prepared by the following method of:

preparation of a compound oil complex: bio-sterilizing and dissolving the fullerene and/or the fullerene derivative into the nutritious compound oil to form the compound oil complex;

preparation of an oil phase: dissolving the emulsifying agent and the optional stabilizer into the compound oil complex to obtain the oil phase;

preparation of an aqueous phase: dissolving the wall material into hot water to obtain the aqueous phase;

mixing of the aqueous phase and the oil phase: mixing and stirring the oil phase and the aqueous phase evenly, and then performing sterilization; and

homogenizing and spray drying: performing homogenizing and spray drying to obtain the micro-capsule powder.

Further, the fullerene and the derivative thereof are capable of increasing beneficial intestinal microorganisms.

Further, the fullerene and the derivative thereof are capable of decreasing harmful intestinal microorganisms.

Further, the fullerene and the derivative thereof are capable of regulating the abundance of intestinal flora.

Further, the fullerene and the derivative thereof are capable of treating or relieving gastrointestinal diseases, neurodegenerative diseases, metabolic diseases, sleep disorders, inflammation, tumors or enhancing immunity by regulating intestinal flora.

Further, the fullerene and the derivative thereof are capable of improving animal physiques, regulating animal intestinal tracts, preventing or treating animal diseases, or promoting animal growth by regulating intestinal flora.

The fullerene and the derivative thereof may increase beneficial bacteria, decrease harmful bacteria and increase the abundance of intestinal flora when used for regulating intestinal flora, which is thus capable of relieving or treating human diseases (including gastrointestinal diseases, neurodegenerative diseases, metabolic diseases, sleep disorders, inflammation, tumors) and enhancing human immunity, improving animal physiques, regulating animal intestinal tracts, preventing or treating animal diseases, or promoting animal growth with great application prospect.

### BRIEF DESCRIPTION OF DRAWINGS

FIG. 1 is a comparison diagram showing diversity of rat intestinal flora in different stages; A is a Shannon index and B is a Chao index.

FIG. 2 is a variation diagram showing relative abundance of rat flora at the level of Phylum in different stages;

FIG. 3 is a variation diagram showing relative abundance of rat flora at the level of Genus in different stages;

FIG. 4 shows influences of fullerene and a derivative thereof on the abundance and diversity of human intestinal flora, where, A is an ACE index result; B is a Chao1 index result; C is a Simpson index result; and D is a Shannon index result;

FIG. 5 shows abundance of human intestinal microecology at the level of Phylum;

FIG. 6 shows abundance of human intestinal microecology at the level of Order;

FIG. 7 shows abundance of human intestinal microecology at the level of Family;

FIG. 8 shows abundance of human intestinal microecology at the level of Genus.

### DESCRIPTION OF EMBODIMENTS

Examples of the present invention will be described in detail below. Specific technologies or conditions not indicated in the examples shall be subjected to the technologies or conditions described in the literature within the art or a product manual. Any used reagent or instrument not marked with manufacturer are all the conventional products capable of being purchased on the market. C₆₀ is provided by Xiamen Funano New Material Technology Co., Ltd.

### Preparation Example 1: Preparation Method of a Fullerene Micro-Capsule Powder C₆₀

(1) Olive oil and flaxseed oil were prepared into a nutritious compound oil according to a ratio of fatty acids of the olive oil to fatty acids of the flaxseed oil of 6:4;

(2) 1 g of fullerene was subjected to C₆₀ sterilization and dissolved into 1 L of nutritious compound oil to form a compound oil complex;

(3) 10 g of sodium caseinate, 5 g of dipotassium phosphate and 15 g of mono/di-glycerin fatty acid ester were dissolved into the compound oil complex to obtain an oil phase;

(4) 500 g of maltodextrin was dissolved into 1 L of 80° C. hot water to obtain an aqueous phase;

(5) the obtained aqueous phase and the oil phase were mixed and stirred evenly, and then sterilized; and

(6) the fullerene micro-capsule powder was obtained by homogenizing and spray drying.

### Example 1: Influences of Fullerene on the Intestinal Flora of Ethanol-Induced Liver Injury Mice

1. Materials and Methods

1.1. Materials:

Fullerene micro-capsule powder prepared in the preparation example 1 (hereinafter referred to as fullerene micro-capsule powder), micro-capsule powder prepared according to the steps of preparation example 1 without the addition of fullerene (hereinafter referred to as blank micro-capsule powder), Kunming mice (male, 4-week old, SPF grade, body weight: 20-22 g, license No.: SCXK (Shanghai) 2017-0005), mice standard fodder, padding (purchased from Minhou County Wu's Laboratory Animals Trading Co., Ltd.), eosin methylene blue agar media, Enterococcus media, MRS agar media, BBL agar media, and Beijing Red Star Erguotou (ABV: 56°).

1.2. Methods:

40 Kunming mice were fed with the standard fodder for one week, and randomly divided into 4 groups by body weight, 10 in each group: blank group (fed with the standard fodder only), model group (fed with the standard fodder only), test group (fed with the fodder containing 10% (mass fraction, similarly hereinafter) of fullerene micro-capsule powder), and solvent control group (fed with the fodder containing 10% of blank micro-capsule powder). The mice were fed in different cages of a same room, received natural lighting, took food and water freely, and environment temperature was controlled within (22±2°) C, and relative humidity was controlled to 60%. The mice in the model group, test group, and the solvent control group were administered intragastrically 1×10⁻³ ml/kg BW Beijing Red Star Erguotou during the feeding process for 8 weeks, then these mice were killed by cervical dislocation and dissected to collect cecal contents of the mice for flora detection. The mice in the blank group were fed with the standard fodder only without the intragastric administration of Beijing Red Star Erguotou during the feeding process, 8 weeks later after feeding, these mice were killed by cervical dislocation and dissected to collect cecal contents of the mice for flora detection.

0.1 g of cecal contents were taken and put to a 5 mL of sterile centrifuge tube, and added with sterile saline solution for dilution according to a ratio of 1:9 (m/V), and then subjected to vortex mixing well to obtain 10⁻¹ diluent. The 10⁻¹ diluent was subjected to gradient dilution until 10⁻⁴, 10-5 and 10⁻⁶ diluent were obtained. 500 ul of three kinds of diluent were respectively taken and coated onto an eosin methylene blue agar medium (Escherichia coli was detected, and cultured for 24 h at 37° C.), an MRS agar medium (Lactobacillus was detected, and cultured for 48 h at 37° C.), an Enterococcus faecalis agar medium (Enterococcus faecalis was detected, and cultured for 48 h at 37° C.), and a BBL agar medium (Bifidobacterium was detected, and anaerobic cultured for 48 h at 37° C.), and 3 parallel tests were performed for each degree of dilution, and the whole process was completed within 30 min. Culture was performed according to the above culture conditions, and then colony counting was performed, and the result was expressed as 1 g (CFU/g). Characteristic bacterial colonies were picked for ecological biochemical experiments first to identify the bacterial genus before colony counting.

2. Statistical Treatment

The statistical analysis of all the data was performed with GraphPad Prism5 and SPSS22.0 software, and the result was represented by “mean value ±standard deviation”, and variance analysis was taken for comparison. p<0.05 shows a significant difference, and p<0.01 shows a highly significant difference.

3. Experimental Result

Experimental results of the influences of fullerene micro-capsule powder on the intestinal flora of ethanol-induced liver injury mice are shown in Table 1.

TABLE 1

| | | | | Enterococcus | |
|---|---|---|---|---|---|
| Group | n | Bifidobacterium | Lactobacillus | faecalis | Escherichia coli |
| Blank group | 10 | 8.10 ± 0.23 | 7.29 ± 0.24 | 6.55 ± 0.28 | 3.35 ± 0.98 |
| Model group | 10 | 6.35 ± 0.11 | 6.21 ± 0.54 | 4.23 ± 0.24 | 4.89 ± 0.78 |
| Test group | 10 | 7.56 ± 0.41^{ab} | 7.18 ± 0.32^{ab} | 6.14 ± 0.32^{ab} | 3.87 ± 0.67^{ab} |
| Solvent control | 10 | 6.38 ± 0.22 | 6.54 ± 0.73^{a} | 5.89 ± 0.12^{a} | 4.76 ± 0.81 |
| group | | | | | |

^{a}is compared with the model group, p < 0.05;
^{b}is compared with the control group, p < 0.05.

It can be seen from Table 1 that the fullerene micro-capsule powder may increase the number of Bifidobacterium, Lactobacillus and Enterococcus faecalis in the intestinal flora of ethanol-induced liver injury mice, and decrease the number of Escherichia coli.

### Example 2: Influences of Fullerene on the Abundance of Rat Intestinal Flora

1. Materials and Methods

1.1 Test Animals

30 SPF-grade rats were selected, and purchased from Minhou County Wu's Laboratory Animals Trading Co., Ltd. The rat standard fodder was purchased from Minhou County Wu's Laboratory Animals Trading Co., Ltd. These rats were randomly divided into 3 groups by body weight: a blank group (fed with the standard fodder), a test group (fed with the fodder containing 10% of fullerene micro-capsule powder), and a control group (fed with the fodder containing 10% of blank micro-capsule powder). These rats were fed at room temperature of 22-24° C. with an illumination period of 12 h/12 h, took food and water freely, and fed in Xiamen University Experimental Animal Center, license No.: SCXK (Zhejiang) 2019-0002. Faeces of the rats were collected respectively on the 4th, 8th and 12th weeks, and then quick-frozen in liquid nitrogen, and preserved in a −80° C. refrigerator for further use.

1.2 DNA Extraction and High-Throughput Sequencing

V3-V4 regions of a bacterium 16S rRNA were subjected to PCR amplification; forward and reverse primers were as follows: 341F:5′-CCTAYGGGRBGCASCAG-3′, 806R:5′-GGACTACNNGGGTATCTAAT-3′. A gene library was constructed for the PCR product, and then IsonS5 XL sequencing platform was taken for bioinformatics analysis on high-quality data. This part was entrusted to Novogene Science and Technology Co., Ltd.; the process was as follows: sample preparation-DNA extraction and detection-PCR amplification-product purification-library preparation and construction-Ion SS™XL test on a machine.

2. Statistical Treatment

Usearch was used to calculate the Operational Taxonomic Unit (OTU) at a 97% similar level; RDP classifier was used to calculate the community composition at each taxonomy level; R language was used to calculate the Alpha diversity of flora (Shannon index and Chao index) and to draw a Rank-abundance curve, where, Rank-abundance curve may perceptually reflect the abundance and uniformity of species. SPSS22.0 software was used to analyze the Shannon index, Chao index of flora as well as differences at the levels of Phylum and Genus. Linear discriminant analysis effect size (LEfSe) was used to estimate the influences of each species abundance on differences between groups and to find out species exerting statistical difference effects on sample partition.

3. Experimental Result

3.1 Analysis on the Diversity of Rat Intestinal Flora

R1 represents the blank group on the 4th week; R2 represents the test group on the 4th week; R3 represents the control group on the 4th week; R4 represents the blank group on the 8th week; R5 represents the test group on the 8th week; R6 represents the control group on the 8th week; R7 represents the blank group on the 12th week; R8 represents the test group on the 12th week; and R9 represents the control group on the 12th week, similarly hereinafter.

Diversity result of rat intestinal flora is shown in FIG. 1, where, A represents a Shannon index, B represents a Chao index and *P<0.05,**P<0.01.

As shown in FIG. 1, on the 4th week, the difference of Shannon indexes of diversity between the test group and the blank group has no statistical significance (P=0.3005), and the difference of Chao indexes has statistical significance (P=0.0192); on the 8th week, the difference of Shannon indexes of diversity between the test group and the blank group has statistical significance (P=0.0466), and the difference of Chao indexes has statistical significance (P=0.0012); on the 12th week, the difference of Shannon indexes of diversity between the test group and the blank group has statistical significance (P=0.0471), and the difference of Chao indexes has statistical significance (P=0.0012).

3.2 Analysis on the Rat Intestinal Flora at the Levels of Phylum and Genus

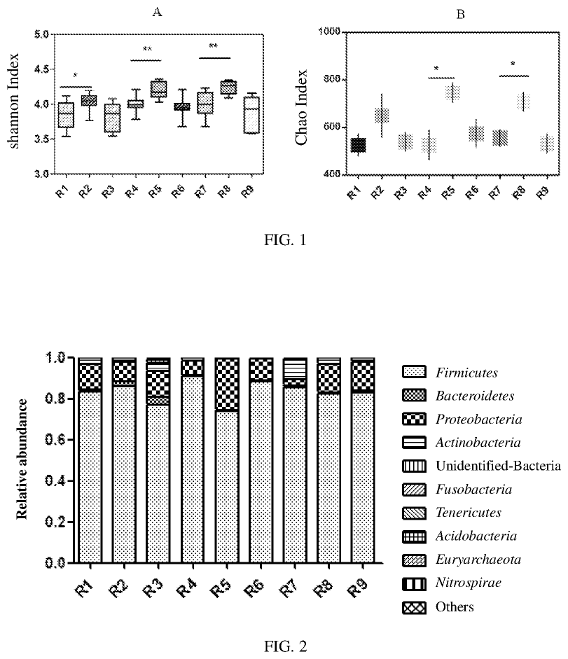

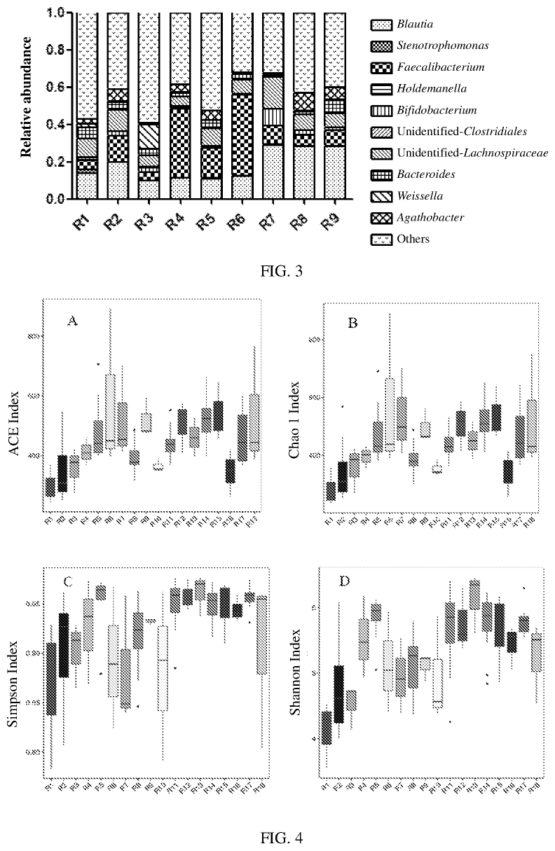

Rankings of the abundance of Phylum and Genus in rat intestinal flora are respectively shown in FIGS. 2 and 3. Each flora in FIG. 2 is as follows: Firmicutes, Bacteroidetes, Proteobacteria, Actinobacteria, Fusobacteria, Tenericutes, Acidobacteria, Euryarchaeota and Nitrospirae. Each flora in FIG. 3 is as follows: Blautia, Stenotrophomonas, Faecalibacterium, Holdemanella, Bifidobacterium, Unidentified-Clostridiales, unidentified-Lachnospiraceae, Bacteroides, Weissella, and Agathobacter).

It can be seen from the results of FIGS. 2 and 3 that for the flora with the abundance of Phylum and Genus ranking the top ten, the Proteobacteria in the test group obviously decreases compared with that of the blank group, and Firmicutes in the test group obviously increases compared with that of the blank group on the 4th, 8th and 12th weeks; on the level of Genus, Stenotrophomonas in the test group obviously decreases compared with that of the blank group; Bifidobacterium and Weissella in the test group obviously increase compared with those of the blank group on the 4th, 8th and 12th weeks.

### Example 3: Influences of Fullerene on the Intestinal Flora of Weaned Piglets

1. Materials and Methods

1.1. Materials:

Superoxide dismutase (SOD) kit, malondialdehyde (MDA) kit, glutathion peroxidase (GSH-Px) kit, fullerene micro-capsule powder, micro-capsule powder, eosin methylene blue agar media and MRS agar media.

1.2 Test animals and daily ration preparation:

60 healthy (body weight: 6.0±0.5 kg) Duroc x Landrace x Yorkshire ternary-hybrid weaned piglets (28 d) were selected and randomly divided into 3 groups, 5 repeats in each group, and 4 piglets in each repeat, and 4 piglets were fed in a pigpen. Test group 1 (blank group): piglets were fed with basic ration; test group 2 (test group): piglets in fullerene micro-capsule powder group were fed with basic ration+0.1% fullerene micro-capsule powder (Example 1); test group 3 (control group): piglets in blank micro-capsule powder group were fed with basic ration+0.1% blank micro-capsule powder. Pre-feeding period was 7 d; formal period was 60 d; and the formal period was divided into 3 stages, and 20 d in each stage. The piglets took food and water freely. The pigpen temperature was kept at 26° C. around. The test animals, basic ration and sites were provided by Xiamen Nongjiayizu Farm.

1.3 Test Method

Food consumption was recorded during the test; the piglets were respectively weighed at 8:00-10:00 at the beginning and end of the test to calculate the average daily gain, average daily feed intake and material-to-weight ratio; the fecal pollution condition and fecal shape at the anus of piglets were carefully observed at 9:00 and 16:00 everyday to record the number of diarrhea piglets and number of diarrhea days in each group, thus calculating the diarrhea rate in each group.

On the same days of the 20 d, the 40 d and the 60 d of the test, 1 healthy piglet with body weight close to mean value was randomly picked from each repeat, and subjected to venous blood sampling, and centrifuged for 15 min at 3000 r/min; blood serum was isolated and kept at −20° C. and to be measured.

On the same days of the 20 d, the 40 d and the 60 d of the test, 1 healthy piglet with body weight close to mean value was randomly picked from each repeat and slaughtered, and laparotomized to take intestinal segments (respectively duodenum, jejunum, ileum, cecum and colon), both ends thereof were ligated, and then immediately brought back to a laboratory for detection. 1 g of intestinal contents were weighed on a clean bench and put to a sterile centrifuge tube, and added with sterile saline solution for dilution according to a ratio of 1:9 (m/V), and then subjected to vortex mixing well to obtain 10⁻¹ diluent. The 10⁻¹ diluent was subjected to gradient dilution until 10⁴, 10-5 and 10⁻⁶ diluent were obtained. 200 ul of the three gradient diluent of duodenum, jejunum, ileum, cecum and colon contents were respectively taken and coated onto eosin methylene blue agar media (Escherichia coli was detected, and cultured for 24 h at 37° C.) and MRS agar media (lactic acid bacteria were detected, and cultured for 48 h at 37° C.), and 3 parallel tests were performed for each degree of dilution. Culture was performed according to the above culture conditions, and then colony counting was performed, and the result was expressed as 1 g (CFU/g). Characteristic bacterial colonies were picked for ecological biochemical experiments first to identify the bacterial genus before colony counting.

2. Data Processing

The statistical analysis of all the data was performed with GraphPad Prism5 and SPSS22.0 software, and the result was represented by “mean value ±standard deviation”, and variance analysis was taken for comparison. p<0.05 shows a significant difference, and p<0.01 shows a highly significant difference.

3. Result

3.1 Influences of Fullerene Micro-Capsule Powder on the Growth Performance and Diarrhea Rate of Weaned Piglets

Test results are shown in Table 2. The calculation way is as follows:

Daily gain: body weight of fasting piglets was weighed at 9:00 in the early morning of the 1st and 60th days during the test, and average daily gain (ADF) during the whole test was calculated; ADF=total weight gain of the pigpen/(number of pigpen piglets x number of test days).

Food consumption: a group was set as a unit during the test to accurately determine and record the daily feeding amount of the fodder, and to calculate the average daily feed intake (ADFI); ADFI=total food consumption of the pigpen/(number of pigpen piglets x number of test days).

Material-to-weight ratio: the material-to-weight ratio (F/G) in each stage is calculated according to a ratio of ADFI in the whole test period to ADG; F/G=ADFI/ADG.

Diarrhea rate (%)=100×[total number of diarrhea piglets in each group during the test period/(total number of piglets in each group x total number of test days)].

TABLE 2
Table for influences of fullerene micro-capsule powder on the growth
performance and diarrhea rate of weaned piglets

| Statistical index | Blank group | Test group | Control group |
|---|---|---|---|
| Mean initial | 6.29 ± 0.05 | 6.33 ± 0.06 | 6.31 ± 0.06 |
| weight/kg | | | |
| Mean final | 35.06 ± 0.42 | 37.82 ± 1.05* | 34.27 ± 0.35 |
| weight/kg | | | |
| ADFI/g | 781.58 ± 7.94 | 780.10 ± 8.62* | 752.83 ± 16.35 |
| ADG/g | 482.54 ± 7.93 | 503.47 ± 13.99 | 466.07 ± 6.62 |
| Material-to-weight | 1.62 ± 0.02 | 1.55 ± 0.02* | 1.61 ± 0.03 |
| ratio | | | |
| Diarrhea rate/% | 6.83 ± 0.21 | 5.67 ± 0.17** | 6.50 ± 0.28 |

Note:
*represents a significant difference (P < 0.05) compared with the blank group;
**represents a highly significant difference (P < 0.001) compared with the blank group; the marker-free represents a non-significant difference (P > 0.05) compared with the blank group.

It can be seen from Table 2 that for mean final weight, there is a significant difference (P=0.0398) between the test group and the blank group; there is a significant difference (P=0.0140) between the test group and the control group; there is no statistic difference between the blank group and the control group; for ADFI, there is a significant difference (P=0.0477) between the test group and the blank group; for the material-to-weight ratio, there is a significant difference (P=0.038) between the test group and the blank group; for the diarrhea rate, there is a highly significant difference (P=0.003) between the test group and the blank group.

3.2 Influences of Fullerene Micro-Capsule Powder on the Antioxidant Activity of Weaned Piglets

Results are Shown in Tables 3-5.

TABLE 3
Table for influences of fullerene micro-capsule powder on the
malondialdehyde content of weaned piglets (nmol/mL)

| Item | Blank group | Test group | Control group |
|---|---|---|---|
| 20 d | 7.06 ± 0.30 | 6.17 ± 0.14 | 6.94 ± 0.42 |
| 40 d | 8.61 ± 0.21 | 6.29 ± 0.09** | 8.42 ± 0.20 |
| 60 d | 8.76 ± 0.14 | 6.43 ± 0.06** | 8.77 ± 0.06 |

Note:
*represents a significant difference (P < 0.05) compared with the blank group;
**represents a highly significant difference (P < 0.001) compared with the blank group; the marker-free represents a non-significant difference (P > 0.05) compared with the blank group.

It can be seen from Table 3 that on the 20th day of the test, there is no significant difference of the malondialdehyde content between the test group and the blank group; on the 40th day of the test, the malondialdehyde content of the test group is significantly lower than that of the blank group (P<0.01); on the 60th day of the test, the malondialdehyde content of the test group is significantly lower than that of the blank group (P<0.01).

TABLE 4
Influences of fullerene micro-capsule powder on the superoxide
dismutase (SOD) activity of weaned piglets (U/mL)

| Item | Blank group | Test group | Control group |
|---|---|---|---|
| 20 d | 103.06 ± 3.85 | 116.04 ± 3.11 | 102.90 ± 5.56 |
| 40 d | 100.94 ± 3.67 | 122.94 ± 6.05* | 96.41 ± 3.53 |
| 60 d | 94.29 ± 4.35 | 129.17 ± 6.83* | 89.62 ± 18.17 |

Note:
*represents a significant difference (P < 0.05) compared with the blank group;
**represents a highly significant difference (P < 0.001) compared with the blank group; the marker-free represents a non-significant difference (P>0.05) compared with the blank group.

It can be seen from Table 4 that on the 20th day of the test, there is no significant difference of the SOD content between the test group and the blank group; on the 40th day of the test, the SOD activity of the test group is significantly higher than that of the blank group (P<0.05); on the 60th day of the test, the SOD activity of the test group is significantly higher than that of the blank group (P<0.05).

TABLE 5
Influences of fullerene micro-capsule powder on the glutathion
peroxidase activity of weaned piglets (U/mL)

| Item | Blank group | Test group | Control group |
|---|---|---|---|
| 20 d | 242.76 ± 7.88 | 262.30 ± 4.28 | 244.53 ± 5.04 |
| 40 d | 169.55 ± 9.96 | 303.59 ± 3.90** | 152.13 ± 11.31 |
| 60 d | 115.49 ± 8.08 | 280.91 ± 14.15** | 119.04 ± 16.90 |

Note:
*represents a significant difference (P < 0.05) compared with the blank group;
**represents a highly significant difference (P < 0.001) compared with the blank group; the marker-free represents a non-significant difference (P>0.05) compared with the blank group.

It can be seen from Table 5 that on the 20th day of the test, there is no significant difference of the GSH-Px content between the test group and the blank group; on the 40th day of the test, the GSH-Px activity of the test group is significantly higher than that of the blank group (P<0.01); on the 60th day of the test, the GSH-Px activity of the test group is significantly higher than that of the blank group (P<0.01).

It can be seen from Tables 2-5 that compared with the blank group, the test group has significant differences in the mean final weight, ADFI, and the material-to-weight ratio, and has highly significant differences in diarrhea rate. At the beginning of the 40th day of the test, the malondialdehyde content decreases significantly; the SOD activity increases significantly and the GSH-Px activity increases significantly.

3.3 Influences of Fullerene Micro-Capsule Powder on the Number of Intestinal Flora of Weaned Piglets

Results are Shown in Tables 6-7.

TABLE 6
Table for influences of fullerene micro-capsule powder on the intestinal
lactic acid bacteria of weaned piglets

| Item | Test stage | Blank group | Test group | Control group |
|---|---|---|---|---|
| Duodenum | 20 d | 3.44 ± 0.05 | 3.68 ± 0.05 | 3.41 ± 0.02 |
| | 40 d | 3.55 ± 0.04 | 4.15 ± 0.05* | 3.55 ± 0.04 |
| | 60 d | 3.51 ± 0.02 | 4.27 ± 0.04** | 3.48 ± 0.06 |
| Jejunum | 20 d | 3.17 ± 0.03 | 3.35 ± 0.05 | 3.16 ± 0.04 |
| | 40 d | 3.25 ± 0.03 | 3.88 ± 0.06* | 3.28 ± 0.02 |
| | 60 d | 3.34 ± 0.08 | 4.12 ± 0.06* | 3.26 ± 0.04 |
| Ileum | 20 d | 2.95 ± 0.05 | 3.07 ± 0.03 | 3.02 ± 0.04 |
| | 40 d | 3.14 ± 0.07 | 3.67 ± 0.06* | 3.20 ± 0.04 |
| | 60 d | 3.10 ± 0.04 | 3.56 ± 0.06* | 3.20 ± 0.03 |
| Cecum | 20 d | 3.62 ± 0.08 | 3.91 ± 0.04 | 3.52 ± 0.03 |
| | 40 d | 3.77 ± 0.04 | 4.43 ± 0.10* | 3.65 ± 0.04 |
| | 60 d | 3.69 ± 0.05 | 4.66 ± 0.06** | 3.71 ± 0.09 |
| Colon | 20 d | 3.71 ± 0.09 | 4.21 ± 0.09* | 3.69 ± 0.10 |
| | 40 d | 3.77 ± 0.11 | 4.43 ± 0.09** | 3.68 ± 0.06 |
| | 60 d | 3.68 ± 0.07 | 4.47 ± 0.05** | 3.42 ± 0.08 |

Note:
*represents a significant difference (P < 0.05) compared with the blank group;
**represents a highly significant difference (P < 0.001) compared with the blank group; the marker-free represents a non-significant difference (P>0.05) compared with the blank group.

It can be seen from Table 6 that compared with the blank group, the number of lactic acid bacteria has no significant difference when the weaned piglets were fed with the fullerene micro-capsule powder for 20 d; after feeding for 40 d, compared with the blank group, the number of lactic acid bacteria increases significantly (P<0.05); contents in each intestinal tract (duodenum, jejunum, ileum, cecum and colon) are consistent; when the weaned piglets were fed for 60 d, compared with the blank group, the number of lactic acid bacteria increases significantly (P<0.05) and contents in each intestinal tract are consistent.

TABLE 7
Table for influences of fullerene micro-capsule powder on the intestinal
Escherichia coli of weaned piglets

| Item | Test stage | Blank group | Test group | Control group |
|---|---|---|---|---|
| Duodenum | 20 d | 2.63 ± 0.02 | 2.70 ± 0.02 | 2.68 ± 0.03 |
| | 40 d | 4.23 ± 0.05 | 2.97 ± 0.05** | 4.24 ± 0.04 |
| | 60 d | 4.78 ± 0.05 | 3.31 ± 0.07** | 4.75 ± 0.06 |
| Jejunum | 20 d | 2.77 ± 0.05 | 2.69 ± 0.06 | 2.69 ± 0.06 |
| | 40 d | 4.43 ± 0.06 | 3.14 ± 0.06* | 4.35 ± 0.15 |
| | 60 d | 5.10 ± 0.05 | 3.12 ± 0.05** | 5.00 ± 0.09 |
| Ileum | 20 d | 2.85 ± 0.10 | 2.95 ± 0.06 | 2.93 ± 0.07 |
| | 40 d | 4.67 ± 0.05 | 3.29 ± 0.06* | 4.60 ± 0.03 |
| | 60 d | 5.22 ± 0.06 | 3.34 ± 0.06** | 5.20 ± 0.05 |
| Cecum | 20 d | 3.01 ± 0.02 | 3.14 ± 0.04 | 3.10 ± 0.02 |
| | 40 d | 4.40 ± 0.03 | 3.24 ± 0.04** | 4.36 ± 0.05 |
| | 60 d | 5.17 ± 0.05 | 3.39 ± 0.10** | 5.26 ± 0.04 |
| Colon | 20 d | 3.14 ± 0.06 | 3.17 ± 0.05 | 3.12 ± 0.05 |
| | 40 d | 4.50 ± 0.07 | 3.24 ± 0.06* | 4.38 ± 0.10 |
| | 60 d | 5.23 ± 0.02 | 3.32 ± 0.10** | 5.18 ± 0.02 |

Note:
*represents a significant difference (P < 0.05) compared with the blank group;
**represents a highly significant difference (P < 0.001) compared with the blank group; the marker-free represents a non-significant difference (P > 0.05) compared with the blank group.

It can be seen from Table 7 that compared with the blank group, the number of Escherichia coli has no significant difference (P>0.05) when the weaned piglets were fed with fullerene micro-capsule powder for 20 d; after feeding for 40 d, compared with the blank group, the number of Escherichia coli has significant differences (P<0.05); contents in each intestinal tract are consistent; when the weaned piglets were fed for 60 d, compared with the blank group, the number of Escherichia coli has highly significant differences (P<0.001) and contents in each intestinal tract are consistent.

It can be seen from Tables 6 and 7 that compared with the blank group, starting from feeding the weaned piglets with fullerene micro-capsule powder for 40 d, the number of lactic acid bacteria in the test group increases significantly, and contents in each intestinal tract (duodenum, jejunum, ileum, cecum and colon) are consistent. The number of Escherichia coli increases significantly and highly significantly increases up to 60 d.

### Example 4 Influences of Fullerene on the Abundance of Human Intestinal Flora

1 Materials and Methods

1.1 Selection for Experimental Samples (Inclusion Criteria)

(1) Healthy people;

(2) free of taking antibiotics and probiotics within nearly 3 months;

(3) exclusion of pregnant women, lactating women and other special population;

(4) no substantial diet change one week before grouping.

1.2 Test Item

From Feb. 19, 2019 to Aug. 1, 2019, six research objects (ZCF, DLT, LXD, HJM, LYM and WCS) began to take the fullerene micro-capsule powder on Apr. 3, 2019 (dosage was 10 g/d/person, and the content of fullerene was 3 mg/d/person) until Jul. 3, 2019.

(1) Body index (BMI value);

(2) blood routine, liver functions and renal function indexes;

(3) analysis on fecal samples:

\textcircled{1} Research Object:

I, specimens selected for the study were fresh fecal samples of people brought into the research;

II, 6 g of naturally discharged fresh feces were taken from a sterile container;

III, each feces specimen was subpackaged into 3 parts according to 2 g; 1 part was subjected to 16SrDNA sequencing analysis, and the rest 2 parts served for further use;

IV, feces specimens were disposed within 2 h and placed into carbon dioxide ice for storage and sending.

\textcircled{2} Test Period and Data Processing:

18 samples were taken for each research object, respectively as follows: 3 samples (respectively on March 4, March 14 and April 1) were taken before administration, 12 samples (respectively on April 4, April 13, April 20, April 28, May 7, May 14, May 22, May 30, June 6, June 13, June 22 and July 1) were taken during the administering process, and 3 samples (July 6, July 12 and July 18) were taken after stopping administration.

Usearch was used to calculate the Operational Taxonomic Unit (OTU) at a 97% similar level, and RDP classifier was used to calculate the community composition at each taxonomy level.

2 Experimental Result

2.1 Results of Body Index (BMI Value) are Shown in Table 8.

TABLE 8

| Item | ZCF | DLT | LXD | HJM | LYM | WCS |
|---|---|---|---|---|---|---|
| February 19 | 29.06 | 25.83 | 27.36 | 24.23 | 24.22 | 23.33 |
| April 3 | 27.99 | 25.83 | 27.36 | 24.29 | 24.39 | 24.84 |
| May 20 | 28.07 | 25.83 | 26.70 | 23.70 | 24.05 | 25.40 |
| June 25 | 28.70 | 25.99 | 26.64 | 24.03 | 24.22 | 25.21 |
| August 1 | 29.01 | 25.83 | 26.70 | 23.46 | 24.05 | 25.59 |

It can be seen from Table 8 that the administration of fullerene micro-capsule powder has no influence on body index.

2.2 Blood Routine, Liver Functions and Renal Function Indexes

Supervision and registration of abnormal indexes prior to not administration are shown in the table below (where, 0307, 0420, 0520, 0625 and 0731 are codes of date, for example, 0307 represents Mar. 7, 2019, and so on; 0307 represents prior to not administration; 0420, 0520 and 0625 represent the process of administration, and 0731 represents after administration).

0520 and 0625 represent the process of administration, and 0731 represents after administration).

| | Reference | | | | | |
|---|---|---|---|---|---|---|
| ZCF | value | 0307 | 0420 | 0520 | 0625 | 0731 |
| Mean corpuscular | 82~100 | 80↓ | 81.2↓ | 82.2 | 80.5↓ | 81.4↓ |
| volume (MCV) | | | | | | |
| Mean corpusular | 316~354 | 356↑ | 356↑ | 347 | 353 | 356↑ |
| hemoglobin | | | | | | |
| concentration (MCHC) | | | | | | |
| SD value of red blood | 38.4~47.0 | 36↓ | 36.3↓ | 37.5↓ | 35.9↓ | 38.2↓ |
| cell distribution width | | | | | | |
| Globulin | 20~40 | 18.7↓ | 25.1 | 21.8 | 23.8 | 24 |
| Albumin/globulin ratio | 1.2~2.4 | 2.57↑ | 1.81 | 2.18 | 1.98 | 1.94 |
| Alanine | 9.0~50 | 60.7↑ | 40.2 | 47.8 | 29.4 | 41.3 |
| aminotransferase | | | | | | |
| Creatinine | 64~104 | 116.5↑ | 114.6↑ | 128.6↑ | 123↑ | 131.21↑ |
| Cystatin C | 0.5~1.07 | 1.14↑ | 1.11↑ | 1.08↑ | 1.10↑ | 1.10↑ |

| | Reference | | | | | |
|---|---|---|---|---|---|---|
| DLT | value | 0307 | 0420 | 0520 | 0701 | 0725 |
| Hemoglobin | 115~150 | 102↓ | 108↓ | 120 | 108↓ | — |
| Hematocrit value | 0.35~0.45 | 0.33↓ | 0.348↓ | 0.361 | 0.343↓ | — |
| (HCT) | | | | | | |
| Mean corpuscular | 82~100 | 78.7↓ | 81.7↓ | 81.9↓ | 87.1 | — |
| volume (MCV) | | | | | | |
| Mean corpuscular | 27~34 | 24.7↓ | 25.4↓ | 27.2 | 27.4 | — |
| hemoglobin (MCH) | | | | | | |
| Mean corpusular | 316~354 | 314↓ | 310↓ | 332 | 315↓ | — |
| hemoglobin | | | | | | |
| concentration (MCHC) | | | | | | |
| CV value of red blood | 11.5~14.5 | 18.5↑ | 19.0↑ | 18↑ | 17.1↑ | — |
| cell distribution width | | | | | | |
| Indirect bilirubin | 5.1~13.7 | 3.4↓ | 7.78 | 6.6 | 5.82 | 9.4 |

| | Reference | | | | | |
|---|---|---|---|---|---|---|
| LXD | value | 0307 | 0420 | 0520 | 0625 | 0731 |
| Lymphocyte ratio | 20~50 | 50.4↑ | 49.4 | 56.4↑ | 53.4↑ | 49.9 |
| Red blood cell count | 4.3~5.8 | 6.24↑ | 6.08↑ | 6.32↑ | 5.99↑ | 6.15↑ |
| Mean corpuscular | 82~100 | 71.5↓ | 72.5↓ | 72.3↓ | 72.3↓ | 72.4↓ |
| volume (MCV) | | | | | | |
| Mean corpuscular | 27~34 | 23.1↓ | 22.9↓ | 22.5↓ | 22.7↓ | 22.6↓ |
| hemoglobin (MCH) | | | | | | |
| SD value of red blood | 38.4~47 | 36.5↓ | 38.3↓ | 39.6 | 37.8↓ | 37.6↓ |
| cell distribution width | | | | | | |
| CV value of red blood | 11.5~14.5 | 14.6↑ | 14.9↑ | 15.6↑ | 14.6↑ | 14.5 |
| cell distribution width | | | | | | |
| Urea nitrogen | 2.8~7.6 | 8.96↑ | 7.51 | 8.58↑ | 5.56 | 6.34 |

| | Reference | | | | | |
|---|---|---|---|---|---|---|
| HJM | value | 0307 | 0420 | 0520 | 0625 | 0731 |
| Monocyte ratio | 3~10 | 10.1↑ | 5.6 | 6.7 | 6.8 | 13.2↑ |
| Monocyte count | 0.1~0.6 | 0.71↑ | 0.44 | 0.37 | 0.4 | 0.6 |
| Creatinine | 49~90 | 43.3↓ | 47.6↓ | 45.4↓ | 43.7↓ | 53.1 |

| | Reference | | | | | |
|---|---|---|---|---|---|---|
| LYM | value | 0307 | 0420 | 0520 | 0625 | 0731 |
| Red blood cell count | 4.3~5.8 | 6.23↑ | 5.64 | 5.90 | 5.84 | 5.67 |
| Hemoglobin | 130~175 | 176↑ | 162 | 164 | 166 | 158 |
| Hematocrit (HCT) | 40~50 | 52.4↑ | 47.9 | 50.10 | 49.5 | 47.8 |
| SD value of red blood | 38.4~47 | 37.4↓ | 37.9↓ | 38.3↓ | 37.7↓ | 36.7↓ |
| cell distribution width | | | | | | |
| Platelet-large cell ratio | 19.7~42.4 | 18.5↓ | 17.6↓ | 19.5↓ | 18.4↓ | 18.8↓ |
| (P-LCR) | | | | | | |
| Glutamyl transpeptidase | 10~60 | 73.2↑ | 57.1 | 63.3↑ | 58.1 | 59.7 |

| | Reference | | | | | |
|---|---|---|---|---|---|---|
| WCS | value | 0307 | 0420 | 0520 | 0625 | 0731 |
| Red blood cell count | 3.8~5.10 | 5.78↑ | 5.94↑ | 5.37↑ | 5.86↑ | 5.66↑ |
| Mean corpuscular | 82~100 | 65.6↓ | 64.5↓ | 64.1↓ | 64.7↓ | 64.5↓ |
| volume (MCV) | | | | | | |
| Mean corpuscular | 27~34 | 20.6↓ | 20.0↓ | 19.6↓ | 18.9↓ | 18.7↓ |
| hemoglobin (MCH) | | | | | | |
| Mean corpusular | 316~354 | 314↓ | 311↓ | 305↓ | 293↓ | 290↓ |
| hemoglobin | | | | | | |
| concentration (MCHC) | | | | | | |
| SD value of red blood | 38.4~47.0 | 38.1↓ | 36.2↓ | 39.2 | 43.4 | 42.2 |
| cell distribution width | | | | | | |
| CV value of red blood | 11.5~14.5 | 16.7 | 16.8 | 17.9 | 20.2 | 19.7 |
| cell distribution width | | | | | | |
| Albumin/globulin ratio | 1.2~2.4 | 1.2 | 1.17↓ | 1.33 | 1.27 | 1.28 |
| Glutamyl transpeptidase | 7.0~45 | 6.3↓ | 7.6 | 6.4↓ | 8.6 | 9.3 |
| Uric acid | 154.7~357.0 | 430.02↑ | 345.78 | 304.17 | 334.87 | 249.91 |

2.3 Analysis on Fecal Samples

2.3.1 Sample Classification Situation

| | Before taking the | In the process of taking | After taking the |
|---|---|---|---|
| | micro-capsule | the micro-capsule | micro-capsule |
| | powder (3 | powder (12 | powder (3 |
| Item | samples) | samples) | samples) |
| ZCF | R1 | R2 | R3 |
| DLT | R4 | R5 | R6 |
| LXD | R7 | R8 | R9 |
| HJM | R10 | R11 | R12 |
| LYM | R13 | R14 | R15 |
| WCS | R16 | R17 | R18 |

R1 is fitted by 3 times of sampling before taking the fullerene micro-capsule powder; R2 is fitted by 12 times of sampling in the process of taking the fullerene micro-capsule powder; R3 is fitted by 3 times of sampling after taking the fullerene micro-capsule powder.

2.3.2 Alpha-Diversity Analysis

Abundance index of flora includes an ACE index and a Chao1 index; the larger the ACE index and the Chao1 index are, the higher the abundance of flora is. Diversity index of flora includes a Shannon index and a Simpson index; the larger the Shannon index and the Simpson index are, the higher the diversity of community is. The obtained results are shown in A-D of FIG. 4.

It can be seen from A-D of FIG. 4 that after taking fullerene, the ACE index and the Chao1 index increase obviously relative to that before taking; the abundance R3>R2>R1; R6>R5>R4; R12>R11>R10; R15>R14>R13; R18>R17>R16; because the classification is subjected to personal distribution, and index clusters a same individual, and has universality among the crowd, it indicates that fullerene may improve the abundance of flora. After taking fullerene, the Shannon index and the Simpson index increase obviously relative to those before taking fullerene; the abundance R2>R3>R1; R5>R4>R6; R8>R9>R7; R11>R12>R10; R17>R18>R16, and experiment comparison is a single factor; therefore, it may indicate that fullerene may increase the diversity of intestinal flora.

2.3.3 Analysis on the Composition and Distribution of Intestinal Microecology

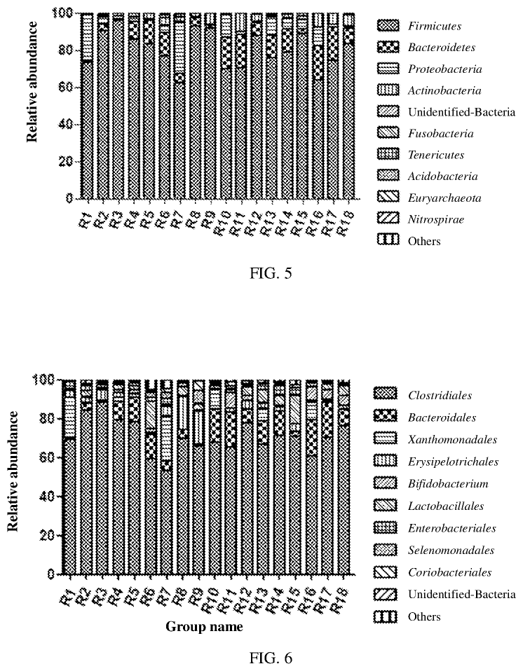

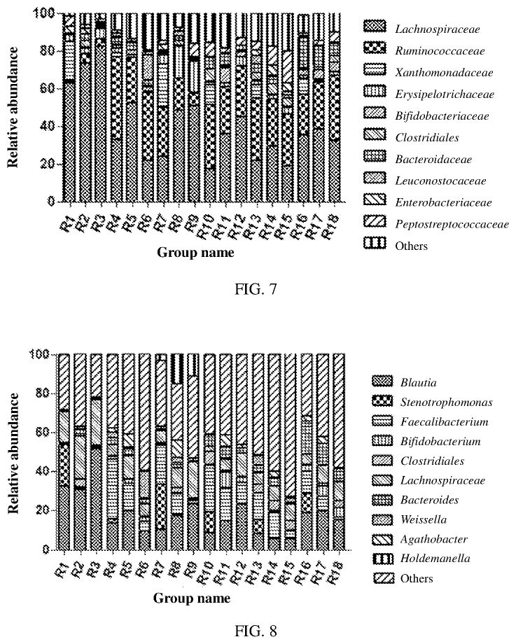

The bacteria whose abundance at the levels of Phylum, Order, Family and Species ranks the top ten were subjected to distribution analysis; where, the distribution result of the flora abundance at the level of Phylumis shown in Table 9 and FIG. 5; the distribution result of the flora abundance at the level of Order is shown in Table 10 and FIG. 6; the distribution result of the flora abundance at the level of Family shown in Table 11 and FIG. 7; the distribution result of the flora abundance at the level of Species is shown in Table 12 and FIG. 8.

Each flora in Table 9 and FIG. 5 is as follows: Nitrospirae, Euryarchaeota, Acidobacteria, Tenericutes, Fusobacteria, Unidentified-Bacteria, Actinobacteria, Bacteroidetes, Proteobacteria, and Firmicutes. Each flora in Table 10 and FIG. 6 is as follows: Unidentified-Bacteria, Coriobacteriales, Selenomonadales, Enterobacteriales, Lactobacillales, Bifidobacterium, Erysipelotrichales, Xanthomonadales, Bacteroidales, and Clostridiales. Each flora in Table 11 and FIG. 7 is as follows: Peptostreptococcaceae, Enterobacteriaceae, Leuconostocaceae, Bacteroidaceae, Unidentified-Clostridiales, Bifidobacteriaceae, Erysipelotrichaceae, Xanthomonadaceae, Ruminococcaceae and Lachnospiraceae. Each flora in Table 12 and FIG. 8 is as follows: Agathobacter, Weissella, Bacteroides, Lachnospiraceae, Clostridiales, Bifidobacterium, Faecalibacterium, Stenotrophomonas, Blautia, and Holdemanella.

TABLE 9

| | | | | | Unidentified- | |
|---|---|---|---|---|---|---|
| Grouping | Firmicutes | Proteobacteria | Bacteroidetes | Actinobacteria | Bacteria | Fusobacteria |
| R1 | 73.48% | 25.31% | 0.30% | 0.63% | 0.13% | 0.14% |
| R2 | 90.11% | 4.01% | 3.70% | 0.96% | 0.55% | 0.10% |
| R3 | 95.88% | 1.66% | 0.86% | 1.33% | 0.18% | 0.02% |
| R4 | 85.83% | 2.49% | 9.34% | 1.95% | 0.01% | 0.00% |
| R5 | 83.40% | 0.69% | 12.64% | 3.01% | 0.04% | 0.03% |
| R6 | 76.89% | 3.83% | 12.66% | 3.87% | 0.08% | 0.01% |
| R7 | 62.37% | 27.77% | 5.08% | 2.55% | 0.04% | 1.66% |
| R8 | 92.87% | 0.62% | 4.59% | 1.86% | 0.02% | 0.01% |
| R9 | 91.96% | 0.49% | 1.17% | 6.10% | 0.04% | 0.01% |
| R10 | 69.91% | 11.68% | 17.00% | 1.16% | 0.03% | 0.01% |
| R11 | 70.41% | 1.20% | 18.11% | 10.12% | 0.02% | 0.01% |
| R12 | 87.65% | 0.33% | 7.19% | 4.33% | 0.02% | 0.01% |
| R13 | 75.86% | 10.08% | 12.03% | 0.93% | 0.04% | 0.00% |
| R14 | 78.83% | 6.16% | 12.56% | 1.71% | 0.05% | 0.01% |
| R15 | 88.86% | 4.67% | 2.67% | 1.93% | 0.35% | 0.01% |
| R16 | 63.92% | 9.82% | 18.61% | 7.46% | 0.01% | 0.00% |
| R17 | 74.25% | 1.26% | 18.17% | 6.06% | 0.01% | 0.01% |
| R18 | 83.26% | 0.79% | 8.66% | 6.27% | 0.05% | 0.01% |

| Grouping | Tenericutes | Acidobacteria | Euryarchaeota | Nitrospirae | Others |
|---|---|---|---|---|---|
| R1 | 0.00% | 0.01% | 0.00% | 0.00% | 0.01% |
| R2 | 0.00% | 0.23% | 0.00% | 0.09% | 0.25% |
| R3 | 0.00% | 0.00% | 0.00% | 0.01% | 0.06% |
| R4 | 0.36% | 0.01% | 0.00% | 0.00% | 0.02% |
| R5 | 0.12% | 0.01% | 0.00% | 0.00% | 0.05% |
| R6 | 1.51% | 0.56% | 0.00% | 0.00% | 0.59% |
| R7 | 0.00% | 0.22% | 0.00% | 0.00% | 0.29% |
| R8 | 0.01% | 0.01% | 0.00% | 0.00% | 0.02% |
| R9 | 0.14% | 0.02% | 0.00% | 0.00% | 0.08% |
| R10 | 0.04% | 0.01% | 0.01% | 0.00% | 0.14% |
| R11 | 0.08% | 0.01% | 0.00% | 0.00% | 0.04% |
| R12 | 0.45% | 0.01% | 0.00% | 0.00% | 0.02% |
| R13 | 0.67% | 0.00% | 0.25% | 0.00% | 0.14% |
| R14 | 0.33% | 0.01% | 0.25% | 0.00% | 0.09% |
| R15 | 1.20% | 0.05% | 0.13% | 0.00% | 0.12% |
| R16 | 0.15% | 0.00% | 0.00% | 0.00% | 0.03% |
| R17 | 0.19% | 0.01% | 0.00% | 0.00% | 0.04% |
| R18 | 0.46% | 0.03% | 0.08% | 0.01% | 0.37% |

TABLE 10

| Grouping | Clostridiales | Bacteroidales | Xanthomonadales | Erysipelotrichales | Bifidobacterium | LactobaciLLales |
|---|---|---|---|---|---|---|
| R1 | 69.34% | 0.30% | 21.27% | 3.36% | 0.44% | 0.74% |
| R2 | 84.42% | 3.63% | 0.27% | 2.74% | 0.52% | 2.72% |
| R3 | 88.48% | 0.72% | 0.08% | 5.54% | 0.94% | 1.79% |
| R4 | 79.40% | 9.34% | 2.10% | 2.54% | 1.51% | 2.59% |
| R5 | 78.25% | 12.63% | 0.13% | 1.68% | 2.21% | 1.78% |
| R6 | 59.57% | 12.47% | 0.07% | 0.54% | 2.24% | 13.96% |
| R7 | 53.53% | 4.69% | 23.08% | 4.87% | 0.48% | 0.47% |
| R8 | 69.75% | 4.59% | 0.19% | 16.69% | 0.51% | 4.75% |
| R9 | 65.62% | 1.15% | 0.03% | 16.86% | 0.87% | 2.88% |
| R10 | 67.70% | 16.98% | 10.53% | 1.72% | 0.43% | 0.46% |
| R11 | 65.32% | 18.08% | 0.10% | 1.92% | 7.88% | 1.72% |
| R12 | 77.62% | 7.18% | 0.00% | 4.57% | 2.65% | 4.48% |
| R13 | 66.77% | 12.02% | 6.77% | 2.33% | 0.34% | 6.42% |
| R14 | 71.41% | 12.55% | 0.09% | 1.96% | 0.68% | 5.08% |
| R15 | 70.87% | 2.63% | 0.03% | 3.71% | 0.31% | 14.16% |
| R16 | 60.60% | 18.56% | 8.93% | 1.12% | 7.12% | 1.86% |
| R17 | 70.16% | 18.14% | 0.67% | 0.67% | 5.13% | 3.12% |
| R18 | 76.42% | 8.63% | 0.01% | 1.82% | 4.98% | 4.94% |

| | | | | Unidentified- | |
|---|---|---|---|---|---|
| Grouping | Enterobacteriales | Selenomonadales | Coriobacteriales | Bacteria | Others |
| R1 | 3.52% | 0.01% | 0.09% | 0.13% | 0.80% |
| R2 | 2.53% | 0.15% | 0.18% | 0.55% | 2.28% |
| R3 | 0.94% | 0.03% | 0.20% | 0.18% | 1.08% |
| R4 | 0.18% | 1.30% | 0.40% | 0.01% | 0.65% |
| R5 | 0.09% | 1.62% | 0.73% | 0.02% | 0.86% |
| R6 | 1.77% | 2.80% | 1.10% | 0.08% | 5.41% |
| R7 | 3.00% | 3.42% | 1.58% | 0.04% | 4.84% |
| R8 | 0.06% | 1.64% | 1.28% | 0.01% | 0.53% |
| R9 | 0.18% | 6.58% | 4.92% | 0.04% | 0.88% |
| R10 | 0.60% | 0.02% | 0.67% | 0.03% | 0.87% |
| R11 | 0.39% | 1.21% | 2.07% | 0.02% | 1.31% |
| R12 | 0.18% | 0.96% | 1.49% | 0.02% | 0.86% |
| R13 | 3.02% | 0.32% | 0.52% | 0.04% | 1.44% |
| R14 | 5.62% | 0.33% | 0.95% | 0.05% | 1.29% |
| R15 | 4.16% | 0.08% | 1.15% | 0.34% | 2.56% |
| R16 | 0.50% | 0.33% | 0.28% | 0.01% | 0.69% |
| R17 | 0.41% | 0.26% | 0.85% | 0.01% | 0.59% |
| R18 | 0.35% | 0.06% | 1.00% | 0.04% | 1.76% |

TABLE 11

| | | | | | | Unidentified- |
|---|---|---|---|---|---|---|
| Grouping | Lachnospiraceae | Ruminococcaceae | Xanthomonadaceae | Erysipelotrichaceae | Bifidobacteriaceae | Clostridiales |
| R1 | 62.69% | 0.95% | 21.27% | 3.36% | 0.44% | 0.53% |
| R2 | 73.29% | 4.80% | 0.27% | 2.74% | 0.52% | 3.91% |
| R3 | 82.12% | 4.18% | 0.08% | 5.54% | 0.94% | 0.60% |
| R4 | 32.77% | 44.04% | 2.10% | 2.54% | 1.51% | 0.37% |
| R5 | 52.34% | 23.92% | 0.13% | 1.68% | 2.21% | 0.23% |
| R6 | 21.84% | 36.07% | 0.05% | 0.54% | 2.24% | 0.46% |
| R7 | 23.83% | 25.92% | 23.08% | 4.87% | 0.48% | 1.31% |
| R8 | 48.48% | 16.73% | 0.19% | 16.69% | 0.51% | 2.61% |
| R9 | 50.71% | 6.86% | 0.03% | 16.86% | 0.87% | 0.77% |
| R10 | 17.27% | 34.04% | 10.53% | 1.72% | 0.43% | 6.41% |
| R11 | 36.02% | 24.84% | 0.09% | 1.92% | 7.88% | 1.05% |
| R12 | 44.66% | 27.36% | 0.00% | 4.57% | 2.65% | 0.64% |
| R13 | 21.73% | 33.19% | 6.77% | 2.33% | 0.34% | 4.18% |
| R14 | 29.36% | 24.94% | 0.08% | 1.96% | 0.68% | 5.27% |
| R15 | 18.71% | 27.42% | 0.02% | 3.71% | 0.41% | 4.69% |
| R16 | 35.13% | 21.52% | 8.93% | 1.12% | 3.12% | 0.98% |
| R17 | 38.42% | 24.97% | 0.67% | 0.67% | 5.13% | 1.48% |
| R18 | 32.23% | 34.62% | 0.01% | 1.82% | 4.98% | 3.55% |

| Grouping | Bacteroidaceae | Leuconostocaceae | Enterobacteriaceae | Peptostreptococcaceae | Others |
|---|---|---|---|---|---|
| R1 | 0.22% | 0.01% | 3.52% | 5.10% | 1.92% |
| R2 | 3.51% | 0.04% | 2.53% | 2.24% | 6.14% |
| R3 | 0.44% | 0.55% | 0.94% | 1.53% | 3.06% |
| R4 | 3.77% | 2.28% | 0.18% | 1.38% | 9.06% |
| R5 | 3.75% | 0.73% | 0.09% | 1.27% | 13.65% |
| R6 | 3.22% | 13.37% | 1.77% | 0.86% | 19.58% |
| R7 | 0.72% | 0.06% | 3.00% | 2.24% | 14.48% |
| R8 | 2.59% | 2.71% | 0.06% | 1.77% | 7.65% |
| R9 | 0.28% | 0.10% | 0.18% | 7.10% | 16.24% |
| R10 | 5.73% | 0.01% | 0.60% | 7.80% | 15.46% |
| R11 | 6.64% | 0.01% | 0.39% | 2.65% | 18.50% |
| R12 | 2.82% | 0.09% | 0.18% | 3.87% | 13.17% |
| R13 | 4.22% | 4.69% | 3.02% | 4.13% | 15.40% |
| R14 | 3.83% | 0.50% | 5.62% | 9.87% | 17.88% |
| R15 | 1.24% | 2.27% | 4.16% | 17.35% | 20.02% |
| R16 | 16.68% | 0.01% | 0.50% | 1.51% | 9.50% |
| R17 | 10.81% | 0.04% | 0.41% | 2.95% | 14.45% |
| R18 | 6.69% | 0.04% | 0.35% | 5.35% | 10.36% |

TABLE 12

| | | | | | Unidentified- |
|---|---|---|---|---|---|
| Grouping | Blautia | Stenotrophomonas | Faecalibacterium | Bifidobacterium | Clostridiales |
| R1 | 32.50% | 21.26% | 0.23% | 0.44% | 0.35% |
| R2 | 30.79% | 0.26% | 0.53% | 0.52% | 3.90% |
| R3 | 51.72% | 0.05% | 0.06% | 0.94% | 0.56% |
| R4 | 13.69% | 2.09% | 30.43% | 1.51% | 0.21% |
| R5 | 19.75% | 0.12% | 14.07% | 2.20% | 0.18% |
| R6 | 9.63% | 0.02% | 4.80% | 2.24% | 0.44% |
| R7 | 10.46% | 23.06% | 19.65% | 0.48% | 1.22% |
| R8 | 17.65% | 0.19% | 10.67% | 0.50% | 2.59% |
| R9 | 23.07% | 0.02% | 1.81% | 0.86% | 0.71% |
| R10 | 8.65% | 10.53% | 24.12% | 0.43% | 6.33% |
| R11 | 14.87% | 0.06% | 16.64% | 7.88% | 0.98% |
| R12 | 23.43% | 0.00% | 10.85% | 2.64% | 0.57% |
| R13 | 8.40% | 6.77% | 13.67% | 0.34% | 4.07% |
| R14 | 5.63% | 0.08% | 13.62% | 0.68% | 5.12% |
| R15 | 5.71% | 0.02% | 4.00% | 0.41% | 4.65% |
| R16 | 18.75% | 8.93% | 11.24% | 3.11% | 0.41% |
| R17 | 19.62% | 0.66% | 7.11% | 5.13% | 1.41% |
| R18 | 15.32% | 0.01% | 1.33% | 4.98% | 3.49% |

| Grouping | Lachnospiraceae | Bacteroides | Weissella | Agathobacter | Others |
|---|---|---|---|---|---|
| R1 | 15.98% | 0.22% | 0.01% | 0.45% | 28.54% |
| R2 | 22.10% | 3.51% | 0.04% | 1.25% | 37.10% |
| R3 | 23.34% | 0.44% | 0.55% | 0.66% | 21.67% |
| R4 | 5.83% | 3.77% | 2.28% | 2.21% | 37.96% |
| R5 | 11.62% | 3.75% | 0.73% | 6.62% | 40.96% |
| R6 | 6.23% | 3.22% | 13.37% | 0.76% | 59.30% |
| R7 | 5.89% | 0.72% | 0.06% | 1.67% | 33.61% |
| R8 | 10.37% | 2.59% | 2.71% | 8.71% | 28.86% |
| R9 | 18.37% | 0.28% | 0.10% | 1.63% | 41.76% |
| R10 | 2.95% | 5.73% | 0.01% | 0.51% | 40.72% |
| R11 | 5.86% | 6.64% | 0.01% | 5.57% | 41.47% |
| R12 | 11.82% | 2.82% | 0.09% | 1.77% | 46.00% |
| R13 | 5.80% | 4.22% | 4.69% | 0.41% | 51.64% |
| R14 | 7.46% | 3.83% | 0.50% | 3.15% | 59.91% |
| R15 | 8.32% | 1.24% | 2.27% | 0.56% | 73.05% |
| R16 | 5.60% | 16.68% | 0.01% | 2.72% | 31.54% |
| R17 | 9.38% | 10.81% | 0.04% | 3.54% | 42.29% |
| R18 | 9.62% | 6.69% | 0.04% | 0.40% | 58.12% |

It can be seen from Tables 9-12 and FIGS. 5-8 that at the level of Phylum, there is a significant difference between Firmicutes and Proteobacteria before and after fullerene is taken by individual, namely, Firmicutes has obviously increased abundance and Proteobacteria has obviously decreased abundance; at the level of Order, Xanthomonadales has significantly decreased abundance after taking fullerene; at the level of Family, Xanthomonadaceae has significantly decreased abundance after taking fullerene, and Bifidobacteriaceae has significantly increased abundance after taking fullerene, and there exists universality among the crowd; at the level of Genus, Stenotrophomonas has significantly decreased abundance after taking fullerene, and Bifidobacterium has significantly increased abundance after taking fullerene, and there exists universality among the crowd.

Even though examples of the present invention have been shown and described above, it should be understood that the above examples are exemplary but are not construed as limiting the present invention. A person skilled in the art may make any change, modification, replacement and transformation on the above examples within the scope of the present invention without departing from the principle and purpose of the present invention.

1. An application of fullerene and a derivative thereof in regulating intestinal flora.

2. The application according to claim 1, wherein the fullerene and the derivative thereof are selected from at least one of C₂ₘ, a fullerene metal derivative, a fullerene oxygen-bearing derivative, or an organic compound-modified or enveloped fullerene; oxygen atoms in the fullerene oxygen-bearing derivative are linked to carbon atoms or bonded to an alkylene chain on a fullerene framework; an organic compound in the organic compound-modified or enveloped fullerene is at least one of cyclodextrin or a crown ether, and 30≤m≤60.

3. The application according to claim 1, wherein the fullerene and the derivative thereof represent as any one of C₂ₘ, M@C₂ₘ, M2@C₂ₘ, MA@C₂ₘ, M3N@C₂ₘ, M2C₂@C₂ₘ, M2S@C₂ₘ, M20@C₂ₘ or M_{X}A_{3-X}N@C₂ₘ, and 30≤m≤60; M and A are each independently selected from any one metallic element of Sc, Y or lanthanide elements, and @ is a linking group or a representing method of a metal.

4. The application according to claim 1, wherein in use procedure, the fullerene and the derivative thereof are used as food, medicaments or health care products in a form of at least one of a tablet, a hard capsule, a soft capsule, an enteric capsule, a micro-capsule powder, granule, a syrup, an injection, an emulsion, a suspending agent, an aerosol, a solution, or an oral or non-oral sustained-release agent, with or without other ingredients added.

5. The application according to claim 1, wherein the fullerene and the derivative thereof configured to increase beneficial intestinal microorganisms.

6. The application according to claim 1, wherein the fullerene and the derivative thereof are configured to decrease harmful intestinal microorganisms.

7. The application according to claim 1, wherein the fullerene and the derivative thereof are configured to regulate abundance of intestinal flora.

8. The application according to claim 1, wherein the fullerene and the derivative thereof are configured to treat or relieve at least one of gastrointestinal diseases, neurodegenerative diseases, metabolic diseases, sleep disorders, inflammation, or tumors or enhance immunity by regulating intestinal flora.

9. The application according to claim 1, wherein the fullerene and the derivative thereof are configured to at least one of improve animal physiques, regulate animal intestinal tracts, prevent or treating animal diseases, or promote animal growth by regulating intestinal flora.

10. The application according to claim 4, wherein the fullerene and the derivative thereof are used in the form of the micro-capsule powder; components of the micro-capsule powder are at least one of fullerene or a fullerene derivative, a nutritious compound oil, an emulsifying agent, a wall material and an optional stabilizer.

11. The application according to claim 10, wherein the nutritious compound oil is a complex of olive oil and flaxseed oil; the emulsifying agent is a complex of sodium caseinate and mono/di-glycerin fatty acid ester; the wall material is maltodextrin, and the optional stabilizer is dipotassium phosphate.

12. The application according to claim 11, wherein each component in the micro-capsule powder is present in the following amount: 1 g of the at least one of fullerene or fullerene derivative, 0.5-1.5 L of the nutritious compound oil, 5-15 g of the sodium caseinate, 10-20 g of the mono/di-glycerin fatty acid ester, 400-600 g of the maltodextrin and 4-6 g of the dipotassium phosphate; a weight ratio of the olive oil to the flaxseed oil in the nutritious compound oil is 5:5-7:3.

13. The application according to claim 10, wherein the micro-capsule powder is prepared by the following method:
preparing a compound oil complex by bio-sterilizing and dissolving the at least one of fullerene or fullerene derivative into the nutritious compound oil to form the compound oil complex;
preparing an oil phase by dissolving the emulsifying agent and the optional stabilizer into the compound oil complex to obtain the oil phase;
preparing an aqueous phase by dissolving the wall material into hot water to obtain the aqueous phase;
mixing the aqueous phase and the oil phase by mixing and stirring the oil phase and the aqueous phase evenly, and then performing sterilization; and
homogenizing and spray drying by performing the homogenizing and the spray drying to obtain the micro-capsule powder.

14. The application according to claim 13, wherein the micro-capsule powder is prepared by the following method:
(1) preparing olive oil and flaxseed oil into the nutritious compound oil according to a ratio of fatty acids of the olive oil to fatty acids of the flaxseed oil of 6:4;
(2) C60 sterilizing and dissolving 1 g of the fullerene into 1 L of the nutritious compound oil to form the compound oil complex;
(3) dissolving 10 g of sodium caseinate, 5 g of dipotassium phosphate and 15 g of mono/di-glycerin fatty acid ester into the compound oil complex to obtain the oil phase;
(4) dissolving 500 g of maltodextrin into 1 L of 80° C. water to obtain the aqueous phase;
(5) mixing and stirring the aqueous phase and the oil phase evenly, and then performing sterilization; and
(6) performing the homogenizing and the spray drying to obtain the micro-capsule powder.

15. A fullerene micro-capsule powder for regulating intestinal flora, wherein components of the fullerene micro-capsule powder are at least one of fullerene or a fullerene derivative, a nutritious compound oil, an emulsifying agent, a wall material, and an optional stabilizer.

16. The fullerene micro-capsule powder for regulating intestinal flora according to claim 15, wherein the nutritious compound oil is a complex of olive oil and flaxseed oil; the emulsifying agent is a complex of sodium caseinate and mono/di-glycerin fatty acid ester; the wall material is maltodextrin, and the optional stabilizer is dipotassium phosphate.

17. The fullerene micro-capsule powder for regulating intestinal flora according to claim 16, wherein each component is present in the fullerene micro-capsule powder in the following amount: 1 g of the at least one of fullerene or fullerene derivative, 0.5-1.5 L of the nutritious compound oil, 5-15 g of the sodium caseinate, 10-20 g of the mono/di-glycerin fatty acid ester, 400-600 g of the maltodextrin and 4-6 g of the dipotassium phosphate.

18. The fullerene micro-capsule powder for regulating intestinal flora according to claim 16, wherein a weight ratio of the olive oil to the flaxseed oil in the nutritious compound oil is 5:5-7:3.

19. A preparation method of the fullerene micro-capsule powder for regulating intestinal flora according to claim 15, wherein the fullerene micro-capsule powder is prepared by the following method:
preparing a compound oil complex by bio-sterilizing and dissolving the at least one of fullerene or fullerene derivative into the nutritious compound oil to form the compound oil complex;
preparing an oil phase by dissolving the emulsifying agent and the optional stabilizer into the compound oil complex to obtain the oil phase;
preparing an aqueous phase by dissolving the wall material into hot water to obtain the aqueous phase;
mixing the aqueous phase and the oil phase by mixing and stirring the oil phase and the aqueous phase evenly, and then performing sterilization; and
homogenizing and spray drying by performing the homogenizing and the spray drying to obtain the fullerene micro-capsule powder.

20. The preparation method of the fullerene micro-capsule powder for regulating intestinal flora according to claim 19, wherein the fullerene micro-capsule powder is prepared by the following method:
(1) preparing olive oil and flaxseed oil into the nutritious compound oil according to a ratio of fatty acids of the olive oil to fatty acids of the flaxseed oil of 6:4;
(2) C60 sterilizing and dissolving 1 g of the fullerene into 1 L of the nutritious compound oil to form the compound oil complex;
(3) dissolving 10 g of sodium caseinate, 5 g of dipotassium phosphate and 15 g of mono/di-glycerin fatty acid ester into the compound oil complex to obtain the oil phase;
(4) dissolving 500 g of maltodextrin into 1 L of 80° C. water to obtain the aqueous phase;
(5) mixing and stirring the aqueous phase and the oil phase evenly, and then performing sterilization; and
(6) performing the homogenizing and the spray drying to obtain the fullerene micro-capsule powder.
